(19) United States
(12) Patent Application Publication
(10) Pub. No.: US 2021/0265071 A1
(43) Pub. Date: Aug. 26, 2021

(54) BEAM-BLOCKING LEAF AND MULTILEAF COLLIMATOR CONTAINING SAME

(71) Applicant: Varian Medical Systems International AG, Steinhausen (CH)

(72) Inventors: Ronan Rochford, Espoo (FI); Ari Harju, Espoo (FI); Juha Kauppinen, Espoo (FI); Timo Ikonen, Espoo (FI)

(21) Appl. No.: 16/801,715

(22) Filed: Feb. 26, 2020

(51) Int. Cl. G21K 1/04; A61N 5/10

(52) U.S. Cl. CPC G21K 1/046; A61N 5/1047

(57) ABSTRACT

A beam-blocking leaf includes a body portion and a head portion. The head portion is movable relative to the body portion, thereby allowing the end surface of the head portion to change an orientation relative to the body portion. A collimator including the beam-blocking leaf and a method of collimating a radiation beam using the collimator are also provided.

### TECHNICAL FIELD

This disclosure relates generally to radiation therapy and imaging. In particular, various embodiments of a beam-blocking leaf and a multileaf collimator containing the beam-blocking leaf are described.

### BACKGROUND

Multileaf collimators (MLCs) are widely used in radiation machines to support various treatments. An MLC includes a plurality of beam-blocking leaves which can be independently moved in and out of a radiation beam to block or shape the beam. The beam-blocking leaves are generally arranged in pairs and disposed in opposing banks. In use, a combined positioning of the beam-blocking leaves may define one or more apertures through which an unblocked radiation beam can pass. A treatment field in an isocenter plane can be then defined by one or more apertures in the MLC, with a size and/or shape generally conforming to the size and/or shape of a target located in the isocenter plane.

Radiation beam penumbra occurs in systems equipped with MLCs at the edges of the treatment field where the radiation intensity decreases with distance from the full intensity region of the field. This phenomenon is a combination of geometric penumbra and transmission penumbra. Geometric penumbra is generally a function of the source size, the distance of the leaves from the source, and the distance of the reference plane from the source. Transmission penumbra is generally a function of material the MLC leaves are made from, the thickness of the leaves, and the energy of the radiation beam. To reduce the undesirable effect of penumbra, the leaf tip of conventional MLC leaves is rounded in order to provide a smooth or uniform penumbra throughout the MCL range. However, a rounded leaf tip does not provide the best possible penumbra performance. Further, the use of a rounded leaf tip, while being capable of solving the problem of varying penumbra, may sacrifice collimation accuracy of the MLC.

Therefore, there is a continuing need for a new beam-blocking leaf design and a multileaf collimator with improved penumbra performance. It would be desirable to provide a leaf tip configuration that can both improve the penumbra performance and maintain the collimation accuracy of the MLC.

### SUMMARY OF THE DISCLOSURE

An embodiment of a beam-blocking leaf comprises a body portion and a head portion. The head portion is movable relative to the body portion, thereby allowing the end surface of the head portion to change an orientation relative to the body portion.

An embodiment of a collimator comprises a first beam-blocking leaf and a second beam-blocking leaf arranged opposed to the first beam-blocking leaf. The first and second beam-blocking leaves are longitudinally movable relative to each other. At least one of the first and second beam-blocking leaves comprises a body portion and a head portion. The head portion is movable relative to the body portion, thereby allowing the end surface of the head portion to change an orientation relative to the body portion.

An embodiment of a method of collimating a radiation beam from a source comprises providing a multileaf collimator (MLC), wherein the MLC comprises a plurality of beam-blocking leaves arranged side by side in a first bank, and a plurality of beam-blocking leaves arranged side by side in a second bank opposed to the first bank, wherein the plurality of beam-blocking leaves in the first bank are longitudinally movable relative to the plurality of beam-blocking leaves in the second bank, forming a plurality of pairs of beam-blocking leaves, and wherein the beam-blocking leaves of at least selected pairs of the plurality of pairs each comprises a body portion and a head portion, wherein the head portion is movable relative to the body portion, thereby allowing the end surface of the head portion to change an orientation relative to the source, and collimating the radiation beam by positioning the plurality of pairs of beam-blocking leaves in the radiation beam, wherein the positioning comprises adjusting the orientation of the end surface of the head portion of at least some of the selected pairs of beam-blocking leaves relative to the source.

This Summary is provided to introduce selected embodiments in a simplified form and is not intended to identify key features or essential characteristics of the claimed subject matter, nor is it intended to be used as an aid in determining the scope of the claimed subject matter. The selected embodiments are presented merely to provide the reader with a brief summary of certain forms the invention might take and are not intended to limit the scope of the invention. Other aspects and embodiments of the disclosure are described in the section of Detailed Description.

### BRIEF DESCRIPTION OF THE DRAWINGS

These and various other features and advantages of the disclosure will become better understood upon reading of the following detailed description and the appended claims in conjunction with the accompanying drawings, where:

FIG. 1 is a simplified illustration of a radiation system according to embodiments of this disclosure.

FIG. 2 is a simplified illustration of a multileaf collimator according to embodiments of this disclosure.

FIG. 3 depicts a side view of an example beam-blocking leaf according to embodiments of the disclosure.

FIG. 4 depicts example moving mechanisms for moving an example beam-blocking leaf according to embodiments of the disclosure.

FIG. 5 is a simplified illustration of a collimator in a side view according to embodiments of the disclosure.

FIG. 6 is a a simplified illustration of a multileaf collimator in a plan view according to embodiments of the disclosure.

FIGS. 7A-7C depict an example method of collimating a radiation beam using an example beam-blocking leaf according to embodiments of the disclosure.

### DETAILED DESCRIPTION OF EMBODIMENTS

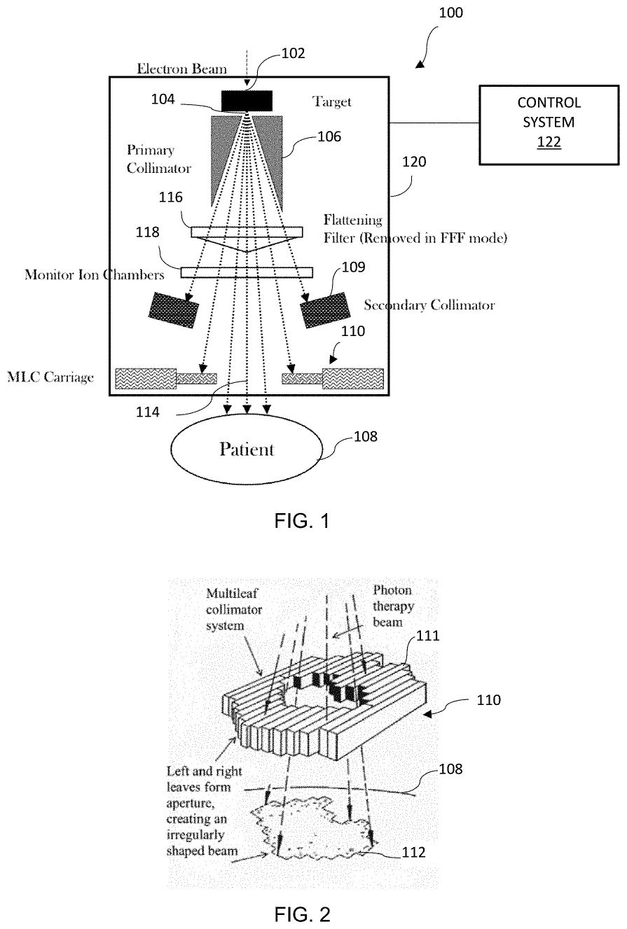

With reference to FIGS. 1-7, various embodiments of a radiation system, a multileaf collimator (MLC), a beam-blocking leaf, and a method of collimating a radiation beam will now be described.

FIG. 1 is a simplified illustration of an example radiation system 100 in which various embodiments of the disclosure can be implemented. As shown, the radiation system 100 includes a radiation source 102 producing or emitting a beam 104 of radiation such as photons, electrons, protons, or other types of radiation. By way of example, the radiation source 102 may include a metallic target configured to produce a beam of x-rays or photons upon impingement of electrons.

The radiation system 100 may also include various collimating devices configured to limit, define, or modify the size, shape, fluence, and other characteristics of the beam. For example, a primary collimator 106 adjacent to the source 102 generally limits the extent of the beam 104 as it travels away from the source 102 toward the patient 108. Motorized secondary collimators or collimation jaws 109 may be included to define the field size. A multileaf collimator (MLC) 110 is disposed between the source 102 and the patient 108 to shape the beam, as indicated by the shaped field 112 shown in FIG. 2. The MLC 110 may be rotated about the central beamline or axis 114, thereby placing the MLC 110 in various orientations. The radiation system 100 may optionally include a flattening filter 116 to provide a uniform beam profile. Alternatively, the radiation system does not include any flattening filter or is flattening-filter-free (FFF) to enhance dose rates for treatment. Ion chamber 118 monitors the parameters of the beam from the source 102.

The source 102, primary collimator 106, secondary collimators 109, MLC 110, and other devices or components may be enclosed in the treatment head 120, which can be rotated by a gantry (not shown) about an axis such as a horizontal axis. Therefore, the system 100 can deliver radiation to a target in the patient 108 from various beam angles. The shape, size, and/or intensity of the beam 104 can be adjusted, or dynamically adjusted, by the MLC 110 as the beam angle is stepped or swept around the target. The operation of the source 102, MLC 110, and other devices can be controlled by a control system 122 such as a treatment delivery system.

The multileaf collimator 110 may be a single level MLC as shown in FIGS. 1 and 2. As used herein, the term “multileaf collimator” or “MLC” refers to a collimation device comprising a plurality of beam-blocking leaves, each of which can be longitudinally moved in and out of a beam to block or shape the beam. Each of the beam-blocking leaves may be driven by a motor with a lead screw or other suitable means. The beam-blocking leaves may be arranged in pairs. The beam-blocking leaves of each pair may be brought in contact or retracted from each other to close or open a path for a radiation beam to pass through the MLC. The beam-blocking leaves may be arranged in opposing banks, or a first bank and a second bank opposing the first bank, and supported by a frame, box, carriage or other support structure, which has features allowing the individual beam-blocking leaves to extend into and retract from the beam. The term “travel range” or “maximal range” may be used herein to refer to the distance between a fully retracted position and a fully extended position of an MLC leaf. The frame, box, carriage or other support structure can be further moved e.g. translated or rotated in addition to the individual leaf travel.

The multileaf collimator 110 may alternatively be a multi-level MLC. By way of example, the MLC 110 may include a first MLC in a first level distal to the source 102 and a second MLC in a second level proximal to the source 102. The first and second MLCs may be arranged such that the moving direction of individual beam-blocking leaves of the first and second MLCs are generally in parallel. Alternatively, the first and second MLCs may be arranged such that the moving direction of the beam-blocking leaves of the first MLC is non-parallel, e.g. perpendicular or at an angle, with respect to the moving direction of the beam-blocking leaves of the second MLC. The first and second MLCs may be arranged such that the leaves of the second MLC may laterally offset the leaves of the first MLC in a top view or as viewed from the source.

In operation, the multileaf collimator 110 may be configured to form an aperture defining a shaped field 112 approximating the target geometry at the isocenter plane. Alternatively, the MLC 110 may be configured to define differently shaped fields at different MLC orientations and/or beam angles, and the doses of multiple fields may be summed to build up a desired dose distribution in the target. Radiation may be delivered intermittently or statically wherein the MLC leaves are in positions while radiation is being delivered. Radiation may also be delivered dynamically wherein the MLC leaves are moving or the MLC is rotating while radiation is being delivered. In some applications, the aperture of the MLC is formed substantially small for small-field radiotherapy such as stereotactic radiosurgery (SRS). By way of non-limiting example, the MLC may be configured to form an aperture defining a field size ranging from 1 to 10 millimeters, or from 4 to 5 millimeters.

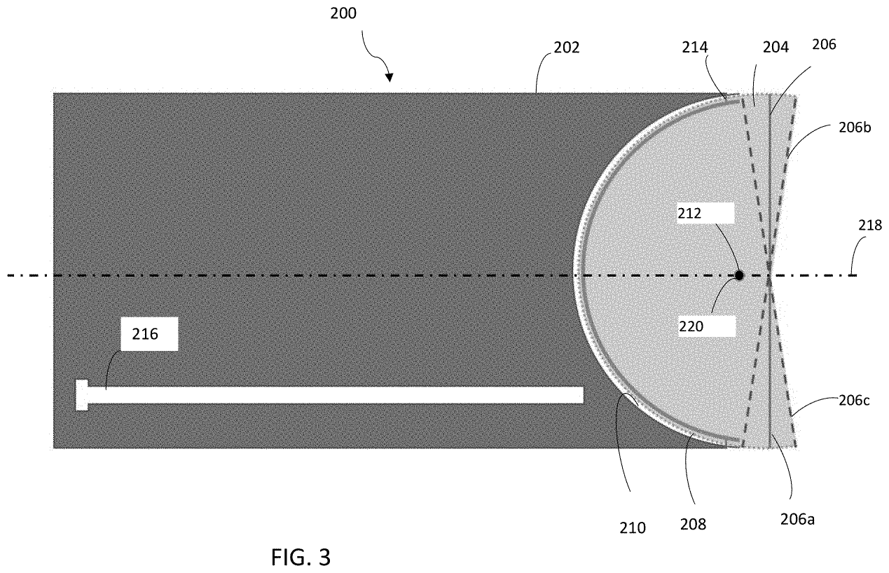

FIG. 3 depicts an example beam-blocking leaf 200 according to embodiments of the disclosure. As shown, the example beam-blocking leaf 200 comprises a body portion 202 and a head portion 204. The head portion 204 includes a tip or end surface 206, which would be inserted into the field when in use. According to embodiments of the disclosure, the head portion 204 is movable relative to the body portion 202, thereby allowing the end surface 206 of the beam-blocking leaf 200 to change an orientation or angle when in use.

The head portion 204 may include a substantially flat end surface 206. As used herein, the term “substantially flat” refers to a surface that is flat or reasonably flat, and is intended to take into consideration small variation that may occur in manufacturing. A flat end surface can provide a desired penumbra performance in shaping the treatment field when the flat surface is aligned with a source or with a divergent beam from the source. As used herein, the phrase “aligned with a source or with a divergent beam from the source” refers to an arrangement or adjustment of the orientation or angle of the flat end surface such that the flat end surface, if extended, intersects the source producing the radiation beam. The source may be a circle or point source or a linear source producing the radiation beam.

It should be noted that the tip or end surface 206 of the beam-blocking leaf 200 may alternatively have various other shapes or configurations according to embodiments of the disclosure. By way of example, the beam-blocking leaf 200 may have a rounded or curved end surface. The beam-blocking leaf 200 may also have an end surface that is a combination of surfaces. The combination of surfaces may include a rounded surface and a flat surface, or may include two or more flat surfaces arranged in angles. The end surface 206 of the beam-blocking leaf 200 may be configured to provide an optimized combination of collimation accuracy and penumbra performance based on clinical applications.

With reference to FIG. 3, the head portion 204 includes a back surface 208. The back surface 208 can be configured to facilitate the movement of the head portion 204 relative to the body portion 202 of the beam-blocking leaf 200. By way of example, the the back surface 208 may be curved. As such, the body portion 202 of the beam-blocking leaf 200 may be provided with a curve or concave surface 210 generally complementary to the curved back surface 208 of the head portion 204 to accommodate and facilitate movement of the head portion 204. In some embodiments, the back surface 208, or at least a portion of the back surface 208, has a rounded or circularly rounded edge profile. For example, as shown in FIG. 3, the back surface 208 may have a constant or substantially constant radius relative to a central point 212. Other shapes and configurations for the back surface are possible to allow the head portion to move relative to the body portion. In some embodiments, the body portion 202 may be provided with a sleeve or groove, or other suitable features 214 that allows the head portion 204 to fit in and be held in place. In some embodiments, the head portion 204 may be held in place via a small pouch (not shown), which can be rotated with the head portion and kept in place in the body portion of the beam-blocking leaf. In some embodiments, the body portion 202 of the beam-blocking leaf 200 may be provided with a channel or hole 216 configured to receive a driving mechanism for moving the beam-blocking leaf 200, as will be described further below.

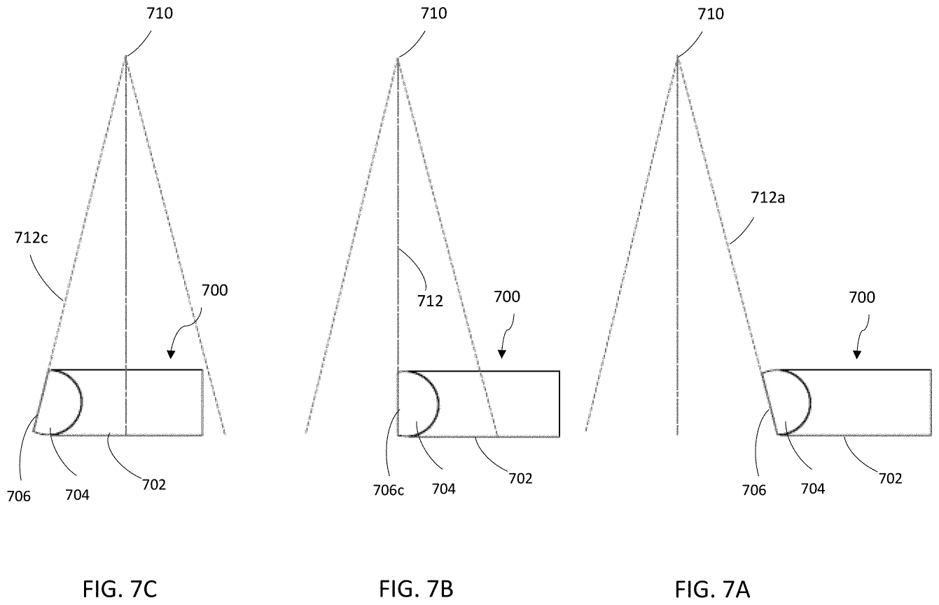

Still with reference to FIG. 3, the head portion 204 may be rotatable about an axis, thereby allowing the orientation or angle of the end surface 206 to change relative to the body portion 202, or relative to a source or a beam from the source when in use. For ease of description, a longitudinal axis 218 is shown on FIG. 3 extending along the body portion 202 or the leaf moving direction. According to embodiments of the disclosure, the head portion 204 is rotatable around an axis that is perpendicular to the longitudinal axis 218, e.g. about an axis 220 through the central point 212. In FIG. 3, line 206a depicts the edge of the end surface 206 at an angle that is perpendicular to the longitudinal axis 218. Line 206b depicts the edge of the end surface 206 at a non-perpendicular angle with respect to the longitudinal axis 218 e.g. when the head portion 204 leans forward. Line 206c depicts the edge of the end surface 206 at another non-perpendicular angle with respect to the longitudinal axis 218 e.g. when the head portion 204 leans backward. Accordingly, the orientation or angle of the end surface 206 of the head portion 204 can be adjusted relative to a source based on the collimation position of the beam-blocking leaf 200 in the field, as will be described further below with reference to FIGS. 7A-7C.

The amount of rotation of the head portion 204 can be determined based on the travel range of the beam-blocking leaf 200. In some embodiments, the head portion 204 can be continuously rotated in a direction, clockwise or counterclockwise, throughout at least a portion of the maximal travel range of the beam-blocking leaf 200. In some embodiment, the head portion 204 can be continuously rotated throughout the entire maximal travel range of the beam-blocking leaf 200. Other considerations in determining the amount of rotation include the elevation of the beam-blocking leaf or leaf bank as measured from the source, the maximal field size, etc. In general, the amount of rotation of the leaf tip or head portion 204 can be determined according to the following equation:

$${tan}^{- 1} ( \frac{\frac{Max Field Size}{2}}{SAD} ) * 2 = Rotation Degrees$$

where SAD represents the source to axis distance or the distance between the source and the axis of rotation of the source, and Max Field Size represents maximal field size at the isocenter plane in a radiation system which includes a collimator comprising the beam-blocking leaf 200.

By way of example, in a system including an MLC placed at an elevation of 510 millimeter (mm), as measured from the source, to provide a maximal field size of 400 mm, where the source to axis (SAD) distance is 1000 mm, an amount of rotation of the tip or head portion 204 in 22.6 degrees would cover the entire travel range of the MLC leaves, as determined by the following equation:

$${tan}^{- 1} ( \frac{\frac{400}{2}}{1000} ) * 2 = 22.6 degrees .$$

As another example, in a system including a multi-level MLC such as a dual-layer MLC to provide a maximal field size of 280 mm, where the source to axis (SAD) distance is 1000 mm, an amount of rotation of the head portion 204 for beam-blocking leaves in the upper MLC (proximal to the source, placed e.g. at 349 mm) in 15.939 degrees would cover the entire travel range of the MLC leaves, as determined by the following equation:

$${tan}^{- 1} ( \frac{\frac{280}{2}}{1000} ) = 7.969 * 2 = 15.939 degrees$$

The amount of rotation of the head portion 204 for the beam-blocking leaves in the lower MLC (distal to the source) would be different as the elevation of the lower MLC leaves is different. In general, the beam-blocking leaf 200 of the disclosure can be constructed to allow the head portion 204 to rotate up to +/−20 degrees, clockwise and/or counterclockwise, to satisfy various clinical applications.

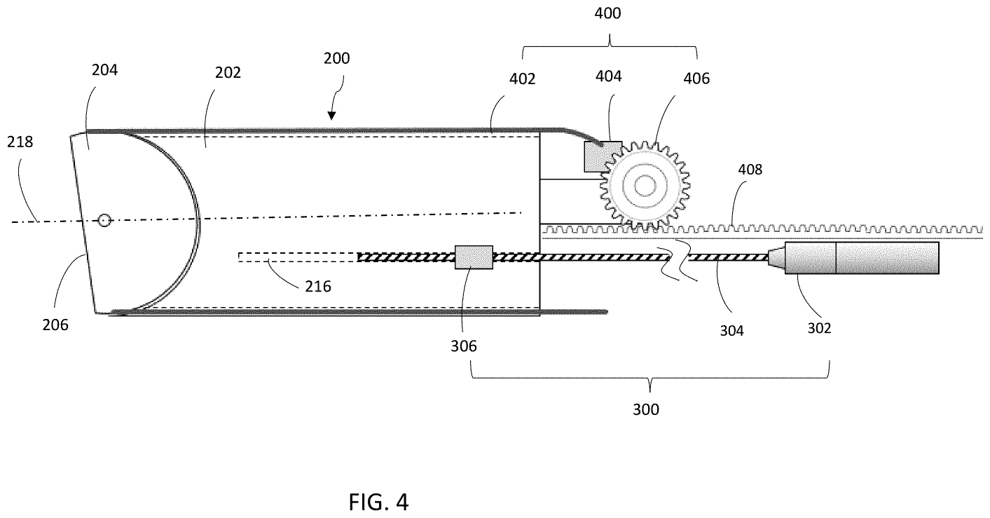

FIG. 4 depicts example mechanisms for moving body portion 202 and head portion 204 of the beam-blocking leaf 200 in accordance with embodiments of the disclosure. It should be noted that the example shown in FIG. 4 is provided for illustration purpose; various other moving mechanisms or ways are possible and can be used with embodiments of the disclosure. The appended claims are not limited to a particular moving mechanism.

As shown in FIG. 4, a first moving mechanism 300 can be used to move or translate the body portion 202 and thus the head portion 204 of the beam-blocking leaf 200 along a longitudinal axis 218. Therefore, the first moving mechanism 300 may function to extend or retract the beam-blocking leaf 200 into or out of a radiation field. The first moving mechanism 300 may include a motor 302 operable to drive or rotate a leaf screw 304. The leaf screw 304 can couple with a leaf nut 306 retained in the body portion 202 of the beam-blocking leaf 200. The rotational torque provided by the drive motor 302 and the leaf screw 304 can be transmitted to the leaf nut 306 and converted to linear force, driving the leaf body portion 202 and head portion 204 in a linear direction, into and/or out of the radiation field. The drive motor 302 can be a servo motor, and a feedback device (not shown) can be coupled to the drive motor to provide feedback on the rotation of the motor so that the position of the leaf screw and thus the position of the beam-blocking leaf can be determined. The first moving mechanism 300 can be controlled by a controller or computer.

The second moving mechanism 400 can be used to move or rotate the head portion 204 relative to the body portion 202 of the beam-blocking leaf 200. Therefore, the second moving mechanism 400 may function to orient or align the tip or end surface 206 of the beam-blocking leaf 200 relative to a source or a divergent beam from the source to provide a best collimation effect and/or penumbra performance. The second moving mechanism 400 may include a flexible wire 402 connecting the head portion 204 of the beam-blocking leaf 200 to a gear box 404. The wire 402, which may lay in a groove, cutout or the like in the body portion 202, may hold the head portion 204 in place and apply rotational torque. The gearbox 404 may include a combination of gears integrated to alter or adjust the torque and speed of the gear 406, and thus the torque and speed applied to the head portion 204 of the beam-blocking leaf 200. The gear 406 may be driven by a drive motor (not shown on FIG. 4) and guided by a guide rail 408. Similar to the first moving mechanism 300, the drive motor for the second moving mechanism 400 can be a servo motor, and a feedback device may be coupled to the drive motor to provide feedback on the rotation of the motor so that the position of the head portion 204 of the beam-blocking leaf 200, or an angle of the end surface 206 of the beam-blocking leaf 200 relative to the source can be determined. The second moving mechanism 400 can be controlled by a controller or computer.

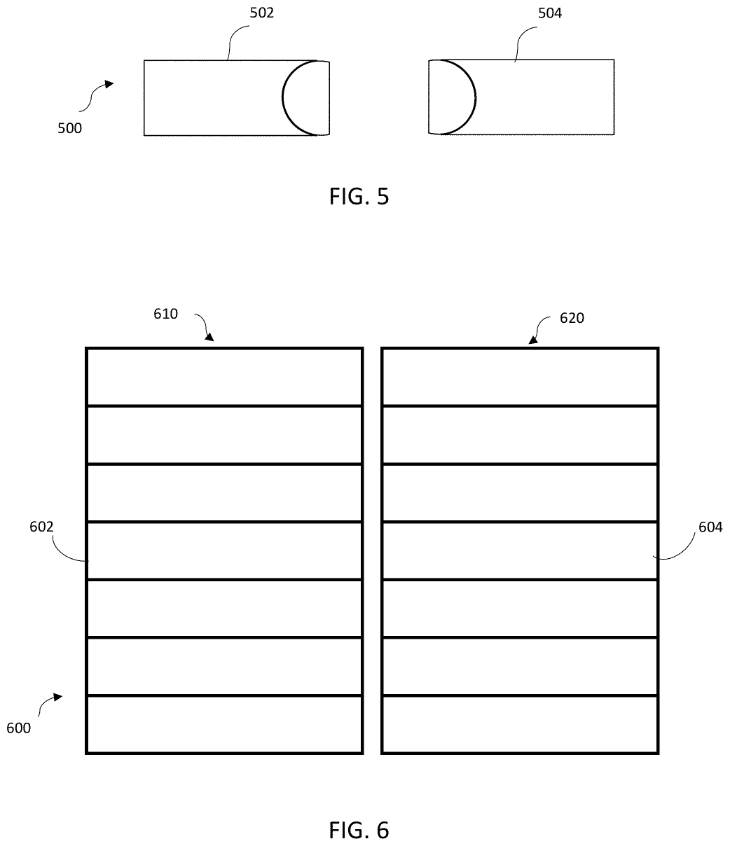

With reference to FIG. 5, in accordance with embodiments of the disclosure, a collimator 500 is provided comprising a first beam-blocking leaf 502 and a second beam-blocking leaf 504. At least one or each of the first and second beam-blocking leaves 502, 504 may have a construction or configuration same as or similar to that of the beam-blocking leaf 200 as described above in connection with FIGS. 3-4. In particular, at least one or each of the first and second beam-blocking leaves 502, 504 comprises a body portion and a head portion, wherein the head portion is movable relative to the body portion, thereby allowing the end surface of the head portion to change an orientation relative to the body portion. The head portion of the at least one or each of the first and second beam-blocking leaves 502, 504 may comprise a substantially flat end surface and a rounded back surface. The body portion of the at least one or each of the first and second beam-blocking leaves 502, 504 may comprise a concave surface generally complementary to the rounded back surface to facilitate a smooth rotation of the head portion. In some embodiments, the rounded back surface of the head portion may have a substantially constant radius. In some embodiments, the head portion of the at least one or each of the first and second beam-blocking leaves 502, 504 is continuously rotatable in a direction throughout the maximal travel range of the at least one or each of the first and second beam-blocking leaves 502, 504. The head portion of the at least one or each of the first and second beam-blocking leaves 502, 504 can be rotatable about an axis up to about ±30 degrees clockwise or counterclockwise. The at least one or each of the first and second beam-blocking leaves 502, 504 may be provided with a first moving mechanism configured to translate the body portion and head portion and a second moving mechanism configured to rotate the head portion.

With reference to FIG. 6, in accordance with embodiments of the disclosure, a multileaf collimator (MLC) 600 is provided comprising a plurality of beam-blocking leaves 602 arranged side by side in a first bank 610 and a plurality of beam-blocking leaves 604 arranged side by side in a second bank 620 opposed to the first bank 610. The plurality of beam-blocking leaves 602 in the first bank 610 are longitudinally movable relative to the plurality of beam-blocking leaves 604 in the second bank 620, forming a plurality of pairs 602/604 of beam-blocking leaves. In some embodiments, beam-blocking leaves of at least one of the plurality pairs may have a construction or configuration same as or similar to that of the example beam-blocking leaf 200 as described above in connection with FIGS. 3-4. In particular, the beam-blocking leaves of at least one pair 602/604 may each include a body portion and a head portion, wherein the head portion is movable relative to the body portion, thereby allowing the end surface of the head portion to change an orientation relative to the body portion. The head portion of beam-blocking leaves of the at least one pair 602/604 may comprise a substantially flat end surface and a rounded back surface. The body portion of beam-blocking leaves of the at least one pair 602/604 may comprise a concave surface generally complementary to the rounded back surface to facilitate a smooth rotation of the head portion. In some embodiments, the rounded back surface of the head portion may have a substantially constant radius. In some embodiments, the head portion of beam-blocking leaves of the at least one pair 602/604 is continuously rotatable throughout the maximal travel range of beam-blocking leaves of the at least one pair. The head portion of beam-blocking leaves of the at least one pair 602/604 can be rotatable about an axis up to about ±30 degrees clockwise or counterclockwise. The beam-blocking leaves of the at least one pair 602/604 may each be provided with a first moving mechanism configured to translate the body portion and a second moving mechanism configured to rotate the head portion of the beam-blocking leaf.

In some embodiments, beam-blocking leaves of more than one pair, or selected pairs, may have a construction or configuration same as or similar to that of the example beam-blocking leaf 200 as described above in connection with FIGS. 3-4. By way of example, beam-blocking leaves of 2-20 pairs, or 2-10 pairs, or 2-4 pairs, each having a construction or configuration same as or similar to that of the example beam-blocking leaf 200 as described above in connection with FIGS. 3-4, may be arranged in or towards the middle section of the MLC 600. In use, the selected pairs of beam-blocking leaves may be used to form an aperture in the MLC defining a field size ranging e.g. from 1 to 10 millimeters or from 4 to 5 millimeters suitable for small-field radiotherapy such as stereotactic radiosurgery (SRS). In some embodiments, beam-blocking leaves of each pair of the MLC may have a construction or configuration same as or similar to that of the example beam-blocking leaf 200 as described above in connection with FIGS. 3-4.

In some embodiments, the plurality of beam-blocking leaves 602, 604 of the MLC 600 may be arranged in multiple levels forming a multi-level MCL, including e.g. a first MLC in a first level and a second MLC in a second level. In some embodiments, beam-blocking leaves of at least one pair in one or each of the first and second MLCs may have a construction or configuration same as or similar to that of the example beam-blocking leaf 200 as described above in connection with FIGS. 3-4. In some embodiments, beam-blocking leaves of some selected pairs in one or each of the first and second MLCs may have a construction or configuration same as or similar to that of the example beam-blocking leaf as described above in connection with FIGS. 3-4. In some embodiments, beam-blocking leaves of each pair in one or each of the first and second MLCs may have a construction or configuration same as or similar to that of the beam-blocking leaf as described above in connection with FIGS. 3-4.

FIGS. 7A-7C illustrate an example method of collimating a radiation beam using a collimator comprising a beam-blocking leaf 700 according to embodiments of the disclosure. It should be noted that the beam-blocking leaf 700 shown in FIGS. 7A-7C can be one of the two beam-blocking leaves in the collimator shown in FIG. 5, such as in the case of collimation jaws. The beam-blocking leaf 700 shown in FIGS. 7A-7C can also be one of the plurality of beam-blocking leaves in the multileaf collimator as shown in FIG. 6. Furthermore, the beam-blocking leaf 700 shown in FIGS. 7A-7C can be one of a plurality of beam-blocking leaves in a multi-level MLC which includes a first MLC distal to the source and a second MLC proximal to the source as discussed above. The beam-blocking leaf 700 may comprise a body portion 702 and a head portion 704 having a substantially flat surface 706. The beam-blocking leaf 700 may have a construction or configuration same as or similar to that of the beam-blocking leaf described above in connection with FIGS. 3-4.

As shown in FIGS. 7A-7C, the beam-blocking leaf 700 of a collimator or MLC may be placed at different positions in shaping a radiation beam based on a treatment plan, for example, at an “under-travel” or first position shown in FIG. 7A, a “central” or second position shown in FIG. 7B, and an “over-travel” or third position shown in FIG. 7C.

With reference to FIG. 7A, the beam-blocking leaf 700 may be placed at the under-travel position by extending from the fully retracted position or by retracting from the central position of the beam blocking leaf using the first moving mechanism 300. At the under-travel position in shaping the field, the head portion 704 of the beam-blocking leaf 700 can be moved or rotated by the second moving mechanism 400, allowing the flat surface 706 of the head portion 704 to lean forward, to align the flat surface 706 to a divergent beam 712a from the source 710, thereby providing an optimal penumbra performance.

With reference to FIG. 7B, the beam-blocking leaf 700 may be placed at the central position by extending from the under-travel position or by retracting from the over-travel position using the first moving mechanism 300. At the central position in shaping the field, the head portion 704 of the beam-blocking leaf 700 can be further moved or rotated by the second moving mechanism 400, allowing the flat surface 706 of the head portion 704 to raise upright, thereby aligning the flat surface 706 to a central beam 712b from the source 710, providing an optimal penumbra performance.

With reference to FIG. 7C, the beam-blocking leaf 700 may be placed at the over-travel position by further extending from the central position using the first moving mechanism 300. At the over-travel position in shaping the field, the head portion 704 of the beam-blocking leaf 700 can be further moved or rotated by the second moving mechanism 400, allowing the flat surface 706 of the head portion 704 of the beam-blocking leaf 700 to lean backward, thereby aligning the flat surface 706 to a divergent beam 712c from the source 710, providing an optimal penumbra performance.

Various embodiments of a beam-blocking leaf, a collimator, and a method of collimating a radiation beam have been described. The new design of the beam-blocking leaf of the disclosure can significantly reduce the penumbra of the field shaped by a collimator including the beam-blocking leaf. The new design of the beam-blocking leaf of the disclosure can provide a smooth or uniform penumbra throughout the collimator range without sacrificing or compromising collimation accuracy.

Various embodiments are described with reference to the figures. It should be noted that some figures are not necessarily drawn to scale. The figures are only intended to facilitate the description of specific embodiments and are not intended as an exhaustive description or as a limitation on the scope of the disclosure. Further, in the figures and description, specific details may be set forth in order to provide a thorough understanding of the disclosure. It will be apparent to one of ordinary skill in the art that some of these specific details may not be employed to practice embodiments of the disclosure. In other instances, well known components may not be shown or described in detail in order to avoid unnecessarily obscuring embodiments of the disclosure.

All technical and scientific terms used herein have the meaning as commonly understood by one of ordinary skill in the art unless specifically defined otherwise. As used in the description and appended claims, the singular forms of “a,” “an,” and “the” include plural references unless the context clearly dictates otherwise. The term “or” refers to a nonexclusive “or” unless the context clearly dictates otherwise. The term “first” or “second” is used to distinguish one element from another in describing various similar elements and should not be construed as in any particular order unless the context clearly dictates otherwise. Relative terms such as “upper,” “above,” “top,” “over,” “on,” “below,” “under,” “bottom,” “lower” or similar terms may be used herein for convenience in describing relative positions or spatial relationships in conjunction with various embodiments. The use of the relative terms should not be construed as to imply a necessary positioning or orientation of the structures or portions thereof in manufacturing or use, and to limit the scope of the invention.

Those skilled in the art will appreciate that various other modifications may be made. All these or other variations and modifications are contemplated by the inventors and within the scope of the invention.

What is claimed is:

1. A beam-blocking leaf comprising a body portion and a head portion including an end surface, wherein the head portion is movable relative to the body portion, thereby allowing the end surface of the head portion to change an orientation relative to the body portion.

2. The beam-blocking leaf of claim 1, wherein the end surface of the head portion comprises a substantially flat surface.

3. The beam-blocking leaf of claim 2, wherein the beam-blocking leaf comprises a longitudinal axis along the body portion, and the head portion is rotatable about an axis generally perpendicular to the longitudinal axis, allowing an angle of the substantially flat surface relative to the longitudinal axis to change.

4. The beam-blocking leaf of claim 3, wherein the head portion is continuously rotatable about the axis clockwise or counterclockwise.

5. The beam-blocking leaf of claim 2, wherein the head portion further comprises a rounded back surface, and the body portion comprises a concave surface generally complementary to the rounded back surface to facilitate rotation of the head portion.

6. The beam blocking leaf of claim 5, wherein the rounded back surface of the head portion has a substantially constant radius.

7. The beam-blocking leaf of claim 1, wherein the end surface of the head portion comprises a slightly rounded surface.

8. A collimator, comprising:
a first beam-blocking leaf and a second beam-blocking leaf arranged opposed to the first beam-blocking leaf, the first and second beam-blocking leaves being longitudinally movable relative to each other, wherein
at least one of the first and second beam-blocking leaves comprises a body portion and a head portion including an end surface, the head portion being movable relative to the body portion, thereby allowing the end surface of the head portion to change an orientation relative to the body portion.

9. The collimator of claim 8, wherein the head portion of the at least one of the first and second beam-blocking leaves comprises a substantially flat end surface and a rounded back surface, and the body portion of the at least one of the first and second beam-blocking leaves comprises a concave surface generally complementary to the rounded back surface to facilitate rotation of the head portion.

10. The collimator of claim 9, wherein the rounded back surface of the head portion has a substantially constant radius.

11. The collimator of claim 9, wherein the at least one of the first and second beam-blocking leaves has a maximal travel range, and the head portion of the at least one of the first and second beam-blocking leaves is continuously rotatable throughout the maximal travel range of the at least one of the first and second beam-blocking leaves.

12. The collimator of claim 9, wherein each of the first and second beam-blocking leaves comprises the head portion and the body portion.

13. The collimator of claim 9, further comprising a first moving mechanism configured to translate the at least one of the first and second beam-blocking leaves, and a second moving mechanism configured to rotate the head portion of the at least one of the first and second beam-blocking leaves.

14. The collimator of claim 8, further comprising:
a plurality of beam-blocking leaves arranged side by side in a first bank,
a plurality of beam-blocking leaves arranged side by side in a second bank opposed to the first bank, wherein the plurality of beam-blocking leaves in the first bank are longitudinally movable relative to the plurality of beam-blocking leaves in the second bank, forming a plurality of pairs of beam-blocking leaves, and wherein
the first beam-blocking leaf is arranged in the first bank and the second beam-blocking leaf is arranged in the second bank forming a pair of beam-blocking leaves longitudinally movable relative to each other.

15. The collimator of claim 14, wherein the head portion of the at least one of the first and second beam-blocking leaves comprises a substantially flat end surface and a rounded back surface, and the leaf body portion of the at least one of the first and second beam-blocking leaves comprises a concave surface generally complementary to the rounded back surface to facilitate rotation of the head portion.

16. The collimator of claim 14, wherein
each of the first and second beam-blocking leaves comprises a body portion and a head portion, the head portion of each of the first and second beam-blocking leaves comprising an end surface and being movable relative to the body portion of corresponding leaf-blocking leaf, thereby allowing the end surface of the head portion of each of the first and second beam-blocking leaves to change an orientation relative to the body portion of corresponding beam-blocking leaf, and
the head portion of each of the first and second beam-blocking leaves comprises a substantially flat end surface and a rounded back surface, and the body portion of each of the first and second beam-blocking leaves comprises a concave surface generally complementary to the rounded back surface of the head portion of corresponding beam-blocking leaf to facilitate rotation of the head portion of each of the first and second beam-blocking leaves.

17. The collimator of claim 16, wherein
each of the plurality of beam-blocking leaves in the first and second banks comprises a body portion and a head portion, the head portion of each of the plurality of beam-blocking leaves in the first and second banks comprises a substantially flat end surface and a rounded back surface, and the body portion of each of the plurality of beam-blocking leaves in the first and second banks comprises a concave surface generally complementary to the rounded back surface of the head portion of corresponding beam-blocking leaf.

18. The collimator of claim 14, wherein the plurality of beam-blocking leaves in the first and second banks are arranged in two or more levels.

19. A method of collimating a radiation beam from a source, comprising:
providing a multileaf collimator (MLC), wherein the MLC comprises:
a plurality of beam-blocking leaves arranged side by side in a first bank, and a plurality of beam-blocking leaves arranged side by side in a second bank opposed to the first bank, wherein the plurality of beam-blocking leaves in the first bank are longitudinally movable relative to the plurality of beam-blocking leaves in the second bank, forming a plurality of pairs of beam-blocking leaves, wherein
beam-blocking leaves of at least selected pairs of the plurality of pairs each comprises a body portion and a head portion, the head portion comprising an end surface and being movable relative to the body portion, thereby allowing the end surface of the head portion to change an orientation relative to the source;
collimating the radiation beam by positioning the plurality of pairs of beam-blocking leaves in the radiation beam, wherein the positioning comprises adjusting the orientation of the end surface of the head portion of at least some of the selected pairs of beam-blocking leaves relative to the source.

20. The method of claim 19, wherein the head portion of the selected pairs of the beam-blocking leaves comprises a substantially flat end surface and a rounded back surface, and the body portion of the selected pairs of the beam-blocking leaves comprises a concave surface generally complementary to the rounded back surface of the head portion, and wherein the adjusting comprises aligning the substantially flat end surface of the head portion of the at least some of the selected pairs of beam-blocking leaves to the source.
